(19) United States
(12) Patent Application Publication
(10) Pub. No.: US 2014/0024613 A1
(43) Pub. Date: Jan. 23, 2014

(54) Methods for Treating HCV

(71) Applicant: AbbVie Inc., North Chicago, IL (US)

(72) Inventors: Daniel E. COHEN, Wilmette, IL (US); Thomas J. PODSADECKI, Northbrook, IL (US); Barry M. BERNSTEIN, Mequon, WI (US); Martin S. KING, Lincolnshire, IL (US)

(21) Appl. No.: 13/927,814

(22) Filed: Jun. 26, 2013

Related U.S. Application Data
(60) Provisional application No. 61/665,019, filed on Jun. 27, 2012.

(51) Int. Cl. A61K 31/7068; A61K 31/497; A61K 31/407; A61K 31/427; A61K 31/4025; A61K 45/06; A61K 31/7056; A61K 31/513

(52) U.S. Cl. CPC A61K 31/7068; A61K 31/7056; A61K 31/497; A61K 31/513; A61K 31/427; A61K 31/4025; A61K 45/06; A61K 31/407
USPC 514/49; 514/43; 514/255.05; 514/274; 514/365; 514/422

(57) ABSTRACT

In one aspect, the present invention features HCV therapies comprising administering to a patient in need thereof an HCV protease inhibitor and ritonavir, wherein ritonavir is used as a pharmacokinetic booster to improve the pharmacokinetics of the HCV protease inhibitor. The HCV therapies do not require the testing of total cholesterol and triglyceride levels prior to and after the therapies.

This application claims priority from U.S. Provisional Application No. 61/665,019, filed Jun. 27, 2012.

### FIELD OF THE INVENTION

The present invention relates to treatment for hepatitis C virus (HCV).

### BACKGROUND

The HCV is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of 3000 amino acids. The polyprotein comprises a core protein, envelope proteins E1 and E2, a membrane bound protein p7, and the non-structural proteins NS2, NS3, NS4A, NS4B, NS5A and NS5B.

Chronic HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often incomplete. Therefore, there is a need for new therapies to treat HCV infection.

### SUMMARY OF THE INVENTION

The present invention features methods of treating HCV with use of ritonavir. Ritonavir is a potent cytochrome P450 3A4 (CYP3A4) inhibitor and can function as a pharmacokinetic booster for drugs that are metabolized by CYP3A4. Numerous HCV protease inhibitors, such as danoprevir and Compound 1 described below, are metabolized by CPY3A4. Co-administration of ritonavir with these HCV protease inhibitors can significantly improve the pharmacokinetics (e.g., AUC or Cₘᵢₙ) of these drugs, leading to less dosing and therefore less side effects associated with these drugs.

Ritonavir, however, has been known to cause elevated cholesterol and triglyceride levels. As a result, the use of ritonavir as a pharmacokinetic booster often requires monitoring total cholesterol and triglyceride levels prior to and after therapy. See, e.g., FDA approved Kaletra® Drug Label as revised in May 2012.

The present invention unexpectedly found that when ritonavir is used to improve the pharmacokinetics of an HCV protease inhibitor, the total cholesterol and triglyceride levels are not elevated. Therefore, monitoring total cholesterol and triglycerides levels prior to and after therapy is not required for these HCV treatments.

Accordingly, in one aspect, the present invention features methods for treating HCV. The methods comprise administering to an HCV patient an effective amount of an HCV protease inhibitor and ritonavir, wherein the total cholesterol and triglyceride levels in said patient are not tested prior to and after the treatment. The HCV protease inhibitor is metabolized by CYP3A4, and ritonavir is used as a pharmacokinetic booster. Ritonavir can, for example and without limitation, be used in an amount of from 100 to 200 mg per dosing. Preferably, ritonavir is used in an amount of 100 mg per co-administration with the HCV protease inhibitor. Preferably, the HCV protease inhibitor is Compound 1 or danoprevir; and more preferably, the HCV protease inhibitor is Compound 1.

In one embodiment of this aspect of the invention, the methods further comprise administering to the patient another anti-HCV agent, such as an HCV NS5A inhibitor, an HCV polymerase inhibitor, an HCV entry inhibitor, a cyclophilin inhibitor, a CD81 inhibitor, or an internal ribosome entry site inhibitor.

In another embodiment, the methods further comprise administering to the patient an HCV NS5A inhibitor or an HCV polymerase inhibitor.

In still another embodiment, the methods further comprise administering to the patient a combination of an HCV NS5A inhibitor and an HCV polymerase inhibitor.

In yet another embodiment, the methods comprise administering the HCV protease inhibitor and ritonavir to the patient at least once a day for no more than 24 weeks (e.g., the treatment duration can be 24, 20, 18, 16, 14 or 12 weeks), wherein the entire treatment regimen does not include administering interferon to the patient. Preferably, the methods further comprise administering to the patient an HCV NS5A inhibitor or an HCV polymerase inhibitor. Also preferably, the methods comprise administering to the patient a combination of an HCV NS5A inhibitor and an HCV polymerase inhibitor.

In yet another embodiment, the methods comprise administering the HCV protease inhibitor and ritonavir to the patient at least once a day for no more than 12 weeks (e.g., the treatment duration can be 12, 10 or 8 weeks), wherein the entire treatment regimen does not include administering interferon to the patient. Preferably, the methods further comprise administering to the patient an HCV NS5A inhibitor or an HCV polymerase inhibitor. Also preferably, the methods comprise administering to the patient a combination of an HCV NS5A inhibitor and an HCV polymerase inhibitor.

In yet another embodiment, the methods comprise administering the HCV protease inhibitor and ritonavir to the patient at least once a day for 12 weeks, wherein the entire treatment regimen does not include administering interferon to the patient. Preferably, the methods further comprise administering to the patient an HCV NS5A inhibitor or an HCV polymerase inhibitor. Also preferably, the methods comprise administering to the patient a combination of an HCV NS5A inhibitor and an HCV polymerase inhibitor.

As a non-limiting example, the HCV protease inhibitor employed in this aspect of the invention or any embodiment thereof can be Compound 1, and said another anti-HCV agent (if used) can be Compound 2. As another non-limiting example, the HCV protease inhibitor employed in this aspect of the invention or any embodiment thereof can be Compound 1, and said another anti-HCV agent (if used) can be Compound 3. As another non-limiting example, the HCV protease inhibitor employed in this aspect of the invention or any embodiment thereof can be Compound 1, and said another anti-HCV agent (if used) can be Compound 4. As another non-limiting example, the HCV protease inhibitor employed in this aspect of the invention or any embodiment thereof can be Compound 1, and said another anti-HCV agent (if used) can be a combination of Compound 2 and Compound 4. As another non-limiting example, the HCV protease inhibitor employed in this aspect of the invention or any embodiment thereof can be Compound 1, and said another anti-HCV agent (if used) can be a combination of Compound 3 and Compound 4. As another non-limiting example, the HCV protease inhibitor employed in this aspect of the invention or any embodiment thereof can be danoprevir, and said another anti-HCV agent (if used) can be mericitabine.

In this aspect of the invention and each embodiment and example thereof, the methods can, for example and without limitation, further comprise administering ribavirin to the patient.

In this aspect of the invention and each embodiment and example thereof, the methods, for example and without limitation, do not comprise administering ribavirin to the patient during the entire treatment regimen.

In another aspect, the present invention features methods of treating HCV using at least two direct-acting antiviral agents (DAAs), wherein one of the two DAAs is an HCV protease inhibitor that is metabolized by CYP3A4, and is co-administered with ritonavir to improve its pharmacokinetics. Preferably, the HCV protease inhibitor is co-formulated with ritonavir in a single composition. The duration of the entire treatment is no more than twelve weeks (e.g., the duration can be 12, 11, 10, 9, or 8 weeks; preferably, the duration of the treatment is 12 weeks). The treatment comprises administering the at least two DAAs to a subject infected with HCV, wherein the total cholesterol and triglyceride levels in the patient are not tested prior to and after the treatment. The treatment does not include administering interferon. The treatment can include administering ribavirin; alternatively, the treatment does not include administering ribavirin. The at least two DAAs can be administered concurrently or sequentially. For example, one DAA can be administered once daily, and another DAA can be administered twice daily. For another example, the two DAAs are administered once daily. For yet another example, the two DAAs together with ritonavir are co-formulated in a single composition and administered concurrently (e.g., once daily). As a non-limiting example, the patient being treated can be infected with HCV genotype 1, such as genotype 1a or 1b. As another non-limiting example, the patient can be infected with HCV genotype 2 or 3. As yet another non-limiting example, the patient can be a HCV-treatment naïve patient, a HCV-treatment experienced patient, an interferon non-responder (e.g., a null responder, a partial responder or a relapser), or not a candidate for interferon treatment. See GUIDANCE FOR INDUSTRY—CHRONIC HEPATITIS C VIRUS INFECTION: DEVELOPING DIRECT-ACTING ANTIVIRAL AGENTS FOR TREATMENT (FDA, September 2010, draft guidance) for the definitions of naive, partial responder, responder relapser (i.e., rebound), and null responder patients.

In another aspect, the present invention features methods of treating HCV using a combination of Compound 1 (or a pharmaceutically acceptable salt thereof) and Compound 2 (or a pharmaceutically acceptable salt thereof), wherein Compound 1 (or the salt thereof) is co-administered with ritonavir. The treatment comprises administering the DAAs to a subject infected with HCV, wherein the total cholesterol and triglyceride levels in the subject are not tested prior to and after the treatment. The duration of the entire treatment regimen is no more than twelve weeks (e.g., the duration can be 12, 11, 10, 9, or 8 weeks; preferably, the treatment lasts for 12 weeks). The treatment does not include administering interferon. The treatment can include administering ribavirin; alternatively, the treatment does not include administering ribavirin. Compound 1 (or the salt thereof) and Compound 2 (or the salt thereof) can be administered concurrently or sequentially. For example, Compound 1 (or the salt thereof) together with ritonavir can be administered once daily, and Compound 2 (or the salt thereof) can be administered twice daily. For another example, Compound 1 (or the salt thereof) together with ritonavir, and Compound 2 (or the salt thereof), are administered once daily. For yet another example, Compound 1 (or the salt thereof) and ritonavir are co-formulated in a single composition and administered concurrently (e.g., once daily). As a non-limiting example, the patient being treated can be infected with HCV genotype 1, such as genotype 1a or 1b. As another non-limiting example, the patient can be infected with HCV genotype 2 or 3. As yet another non-limiting example, the patient can be a HCV-treatment naïve patient, a HCV-treatment experienced patient, an interferon non-responder (e.g., a null responder), or not a candidate for interferon treatment.

In another aspect, the present invention features methods of treating HCV using a combination of Compound 1 (or a pharmaceutically acceptable salt thereof) and Compound 3 (or a pharmaceutically acceptable salt thereof), wherein Compound 1 (or the salt thereof) is co-administered with ritonavir. The treatment comprises administering the DAAs to a subject infected with HCV, wherein the total cholesterol and triglyceride levels in the subject are not tested prior to and after the treatment. The duration of the treatment regimen is no more than twelve weeks (e.g., the duration can be 12, 11, 10, 9, or 8 weeks; preferably, the treatment lasts for 12 weeks). The treatment does not include administering interferon. The treatment can include administering ribavirin; alternatively, the treatment does not include administering ribavirin. Compound 1 (or the salt thereof) and Compound 3 (or the salt thereof) can be administered concurrently or sequentially. For example, Compound 1 (or the salt thereof) together with ritonavir can be administered once daily, and Compound 3 (or the salt thereof) can be administered twice daily. For another example, Compound 1 (or the salt thereof) together with ritonavir, and Compound 3 (or the salt thereof), are administered once daily. For yet another example, Compound 1 (or the salt thereof) and ritonavir are co-formulated in a single composition and administered concurrently (e.g., once daily). As a non-limiting example, the patient being treated can be infected with HCV genotype 1, such as genotype 1a or 1b. As another non-limiting example, the patient can be infected with HCV genotype 2 or 3. As yet another non-limiting example, the patient can be a HCV-treatment naïve patient, a HCV-treatment experienced patient, an interferon non-responder (e.g., a null responder), or not a candidate for interferon treatment.

In another aspect, the present invention features methods of treating HCV using a combination of Compound 1 (or a pharmaceutically acceptable salt thereof) and Compound 4 (or a pharmaceutically acceptable salt thereof), wherein Compound 1 for the salt thereof) is co-administered with ritonavir. The treatment comprises administering the DAAs to a subject infected with HCV, wherein the total cholesterol and triglyceride levels in the subject are not tested prior to and after the treatment. The duration of the treatment regimen is no more than twelve weeks (e.g., the duration can be 12, 11, 10, 9, or 8 weeks; preferably, the treatment lasts for 12 weeks). The treatment does not include administering interferon. The treatment can include administering ribavirin; alternatively, the treatment does not include administering ribavirin. Compound 1 (or the salt thereof) and Compound 4 (or the salt thereof) can be administered concurrently or sequentially. For example, Compound 1 (or the salt thereof) together with ritonavir can be administered once daily, and Compound 4 (or the salt thereof) can be administered twice daily. For another example, Compound 1 (or the salt thereof) together with ritonavir, and Compound 4 (or the salt thereof), are administered once daily. For yet another example, Compound 1 (or the salt thereof) and ritonavir are co-formulated in a single composition and administered concurrently (e.g., once daily). For yet another example, Compound 1 (or the salt thereof), ritonavir, and Compound 4 (or the salt thereof) are co-formulated in a single composition and administered concurrently (e.g., once daily). As a non-limiting example, the patient being treated can be infected with HCV genotype 1, such as genotype 1a or 1b. As another non-limiting example, the patient can be infected with HCV genotype 2 or 3. As yet another non-limiting example, the patient can be a HCV-treatment naïve patient, a HCV-treatment experienced patient, an interferon non-responder (e.g., a null responder), or not a candidate for interferon treatment.

In another aspect, the present invention features methods of treating HCV using a combination of Compound 1 (or a pharmaceutically acceptable salt thereof). Compound 2 (or a pharmaceutically acceptable salt thereof), and Compound 4 (or a pharmaceutically acceptable salt thereof), wherein Compound 1 (or the salt thereof) is co-administered with ritonavir. The treatment comprises administering the DAAs to a subject infected with HCV, wherein the total cholesterol and triglyceride levels in the subject are not tested prior to and after the treatment. The duration of the treatment regimen is no more than twelve weeks (e.g., the duration can be 12, 11, 10, 9, or 8 weeks; preferably, the treatment lasts for 12 weeks). The treatment does not include administering interferon. The treatment can include administering ribavirin; alternatively, the treatment does not include administering ribavirin. Compound 1 (or the salt thereof), Compound 2 (or the salt thereof), and Compound 4 (or the salt thereof) can be administered concurrently or sequentially. For example, Compound 1 (or the salt thereof) together with ritonavir can be administered once daily, and Compound 4 (or the salt thereof) can be administered once daily, and Compound 2 (or the salt thereof) can be administered twice daily. For another example, Compound 1 (or the salt thereof) together with ritonavir, Compound 2 (or the salt thereof), and Compound 4 (or the salt thereof) are administered once daily. For yet another example, Compound 1 (or the salt thereof), ritonavir, and Compound 4 (or the salt thereof) are co-formulated in a single composition and administered concurrently (e.g., once daily). As a non-limiting example, the patient being treated can be infected with HCV genotype 1, such as genotype 1a or 1.b. As another non-limiting example, the patient can be infected with HCV genotype 2 or 3. As yet another non-limiting example, the patient can be a HCV-treatment naïve patient, a HCV-treatment experienced patient, an interferon non-responder (e.g., a null responder), or not a candidate for interferon treatment.

In another aspect, the present technology features methods of treating HCV using a combination of danoprevir and mericitabine, wherein danoprevir is co-administered with ritonavir. The treatment comprises administering the DAAs to a subject infected with HCV, wherein the total cholesterol and triglyceride levels in the subject are not tested prior to and after the treatment. The duration of the treatment regimen is no more than twelve weeks (e.g., the duration can be 12, 11, 10, 9, or 8 weeks; preferably, the treatment lasts for 12 weeks). The treatment does not include administering interferon. The treatment can include administering ribavirin: alternatively, the treatment does not include administering ribavirin. The at least two DAAs can be administered concurrently or sequentially. For example, danoprevir together with ritonavir can be administered once daily, and mericitabine can be administered twice daily. For another example, danoprevir together with ritonavir, and mericitabine, are administered once daily. For yet another example, danoprevir and ritonavir are co-formulated in a single composition and administered concurrently (e.g., once daily). As a non-limiting example, the patient being treated can be infected with HCV genotype 1, such as genotype 1a or 1b. As another non-limiting example, the patient can be infected with HCV genotype 2 or 3. As yet another non-limiting example, the patient can be a HCV-treatment naive patient, a HCV-treatment experienced patient, an interferon non-responder (e.g., a null responder), or not a candidate for interferon treatment.

In any aspect of the invention and each embodiment and example thereof, ritonavir can be readily replaced with cobicistat.

In any aspect of the invention and each embodiment and examples thereof, the testing for the total cholesterol and triglyceride levels can be absent during the treatment, instead of prior to and after the treatment.

Other features, objects, and advantages of the present invention are apparent in the detailed description that follows. It should be understood, however, that the detailed description, while indicating preferred embodiments of the invention, are given by way of illustration only, not Various changes and modifications within the scope of the invention will become apparent to those skilled in the art from the detailed description

### BRIEF DESCRIPTION OF THE DRAWINGS

The drawings are provided for illustration, not limitation.

FIG. 1 shows total cholesterol and triglyceride changes after 48-week HIV therapy as compared to after 12-week HCV therapy.

### DETAILED DESCRIPTION

As used herein, Compound 1 refers to (2R,6S,13aS,14aR,16aS,Z)-N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a ,151616a-hexadecahydrocyclopropa[e]pyrrolo[1,2-a] [1,4]diazacyclopentadecine-14a-carboxamide. Compound 1 is a potent HCV protease inhibitor. The synthesis and formulation of Compound 1 are described in U.S. Patent Application Publication Nos. 2010/0144608 and 2011/0312973, both of which are incorporated herein by reference in their entireties. When co-administered with ritonavir, Compound 1 or a pharmaceutically acceptable salt thereof can be used in any suitable amount such as, for example, in a total daily dose amount of from 50 mg to 250 mg, preferably from 100 mg to 250 mg. For example, Compound 1 or a pharmaceutically acceptable salt thereof can be used in a total daily dose amount of 50, 75, 100, 125, 150, 175, 200, 225 or 250 mg, or any suitable amounts there between.

As used herein, Compound 2 refers to

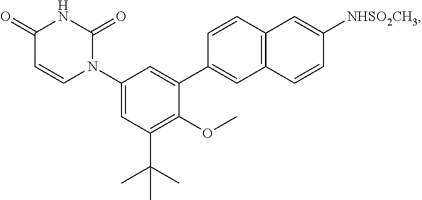

or N-(6-(3-tert-butyl-5-(2,4-dioxo-3,4-dihydropyrimidin-1(2H)-yl)-2-methoxyphenyl)naphthalen-2-methanesulfonamide. Compound 2 and its pharmaceutically acceptable salts are described in International Publication No. WO2009/039127. Compound 2 or a pharmaceutically acceptable salt thereof can be administered in any suitable amount such as, for example, in a total daily dose amount of from 300 mg to 1800 mg, or from 400 mg to 1600 mg, or from 600 mg to 1800 mg, or from 800 mg to 1600 mg or any amounts there between. In some embodiments, Compound 2 or a pharmaceutically acceptable salt thereof can be administered in a total daily dose amount from 100 mg to 800 mg, preferably form 200 mg to 800 mg. In some embodiments, the total daily dosage amount for Compound 2 is 100 mg. In some embodiments, the total daily dosage amount for Compound 2 is 200 mg. In some embodiments, the total daily dosage amount for Compound 2 is 300 mg. In some embodiments, the total daily dosage amount for Compound 2 is 400 mg. In some embodiments, the total daily dosage amount for Compound 2 is 600 mg. In some embodiments, the total daily dosage amount for Compound 2 is 800 mg. In some embodiments, the total daily dosage amount for Compound 2 is 1200 mg. In some embodiments, the total daily dosage amount for Compound 2 is 1600 mg.

As used herein, Compound 3 refers to

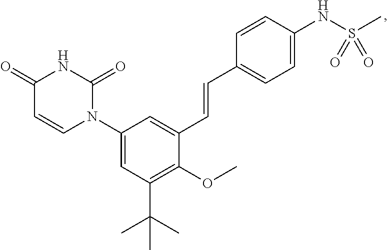 [SMILES: COc1c(/C=C/c2ccc(NS(C)(=O)=O)cc2)cc(-n2ccc(=O)[nH]c2=O)cc1C(C)(C)C]

or (E)-N-(4-(3-tert-butyl-5-(2,4-dioxo-3,4-dihydropyrimidin-1(2H)-yl)-2-methoxystyryl)phenyl)methanesulfonamide. Compound 3 and its pharmaceutically acceptable salts are described in International Publication No. WO2009/039127. For example and without limitation. Compound 3 or a pharmaceutically acceptable salt thereof can be administered in a total daily dose amount of from 50 mg to 1000 mg or from 100 mg to 600 mg or from 80 mg to 320 mg or any amounts there between. In some embodiments, the total daily dosage amount for Compound 3 is 50 mg. In some embodiments, the total daily dosage amount for Compound 3 is 80 mg. In some embodiments, the total daily dosage amount for Compound 3 is 100 mg. In some embodiments, the total daily dosage amount for Compound 3 is 160 mg. In some embodiments, the total daily dosage amount for Compound 3 is 300 mg. In some embodiments, the total daily dosage amount for Compound 3 is 320 mg. In some embodiments, the total daily dosage amount for Compound 3 is 400 mg. In some embodiments, the total daily dosage amount for Compound 3 is 600 mg.

As used herein, Compound 4 refers to

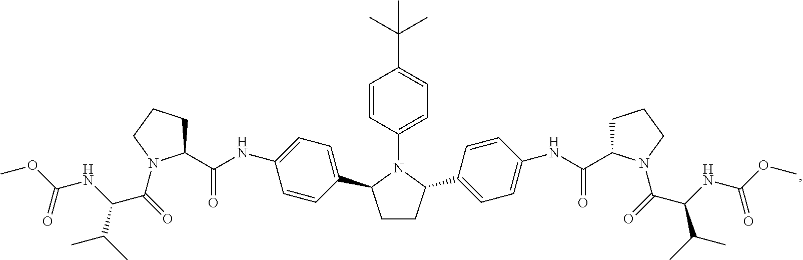

or dimethyl (2S,2′S)-1,1′-((2S,2S)-2,2′(4,4′-((2S,5S)-1-(4-tert-butylphenyl)pyrrolidine-2,5,diyl)bis(4,1-phenylene))bis(azanediyl)bis(oxomethylene)bis(pynolidine-2,1-diyl)bis(3-methyl-1-oxobutane-2,1-diyl)dicarbamate. Compound 4 is described in U.S. Publication No. 2010/0317568, which is incorporated herein by reference. As non-limiting examples, Compound 4 or a pharmaceutically acceptable salt thereof can be administered in a total daily dose amount of from 5 mg to 300 mg, or from 25 mg to 200 mg, or from 25 mg to 50 mg or any amounts there between. In some embodiments, the total daily dosage amount for Compound 4 is 25 mg. In some embodiments, the total daily dosage amount for Compound 4 is 5 mg, alternatively 10 mg, alternatively 20 mg, alternatively 25 mg, alternatively 30 mg, alternatively 35 mg, alternatively 40 mg, or alternatively 50 mg.

DAAs suitable for the present invention include, but are not limited to, HCV protease inhibitors, HCV polymerase inhibitors, HCV NS5A inhibitors, HCV entry inhibitors, cyclophilin inhibitors, CD81 inhibitors, or internal ribosome entry site inhibitors. An HCV polymerase inhibitor can be, for example, a nucleoside polymerase inhibitor, a nucleotide polymerase inhibitor, a non-nucleoside polymerase inhibitor, or a non-nucleotide polymerase inhibitor.

Any suitable form or formulation of ribavirin may be employed in the present invention. Exemplary formulations of ribavirin include COPEGUS®, REBETOL® and RIBASPHERE®. An exemplary pro-drug of ribavirin is taribavirin having the chemical name a 1-β-D-ribofuranosyl-1,2,4-triazole-3-carboxamidine. Ribavirin and taribavirin can be administered in accordance with ribavirin and taribavirin administration well known in the art. For example, COPEGUS® or REBETOL® can be administered in a daily dosage amount of from 500 mg to 1500 mg in one dose or in divided doses. In some embodiments. COPEGUS® or REBETOL® is administered in a daily dosage amount of 800 mg. In some embodiments, REBETOL® is administered in a daily dosage amount of 1000 mg. In some embodiments. COPEGUS® or REBETOL® is administered in a daily dosage amount of 1200 mg. In some embodiments. REBETOL® is administered in a daily dosage amount of 1400 mg. Suitable dosages of ribavirin are dependent on the weight of the subject, for example 1000-1200 mg. Suitable total daily dosages of ribavirin include, but are not limited to 400 mg to 1400 mg a day, alternatively 800 mg to 1400 mg per day, alternatively 400 mg to 1200 mg, alternatively 800 mg to 1200 mg.

The current standard of care (SOC) for the treatment of HCV includes a course of treatment of interferon, e.g. pegylated interferon (e.g., pegylated interferon-alpha-2a or pegylated interferon-alpha-2b, such as PEGASYS by Roche, or PEG-INTRON by Schering-Plough), together with ribavirin (e.g., COPEGUS by Roche, RE:BETOL by Schering-Plough, or RIBASPHERE by Three Rivers Pharmaceuticals). The treatment often lasts for 24-48 weeks, depending on hepatitis C virus genotype. Other interferons include, but are not limited to, interferon-alpha-2a (e.g., Roferon-A by Roche), interferon-alpha-2b (e.g., Intron-A by Schering-Plough), and interferon alfacon-1 (consensus interferon) (e.g., Infergen by Valeant).

The interferon/ribavirin-based treatment is often physically demanding, and can lead to temporary disability in some cases. A substantial proportion of patients will experience a panoply of side effects ranging from a “flu-like” syndrome (the most common, experienced for a few days after the weekly injection of interferon) to severe adverse events including anemia, cardiovascular events and psychiatric problems such as suicide or suicidal ideation. The latter are exacerbated by the general physiological stress experienced by the patients. The present invention allows effective treatment of HCV infection without the use of interferon and also for a shorter period of time, such as a treatment duration of no more than 12 weeks.

In one aspect, the present invention features a method of treating HCV using a combination of two or more DAAs, wherein one of the DAAs is an HCV protease inhibitor that is metabolized by CYP3A4. The HCV protease inhibitor is co-administered with ritonavir to improve its pharmacokinetics. The method comprises administering to a patient in need thereof an effective amount of a combination of the DAAs, wherein the total cholesterol and triglyceride levels in the patient are not tested prior to and after the treatment. The duration of the entire treatment lasts for no more than 24 weeks; for example, the duration of the treatment lasts for 24, 23, 22, 21, 20, 19, 18, 17, 16, 15, 14, 13, 12, 11, 10, 9, or 8 weeks; preferably, the duration of the treatment lasts for 12 weeks. Lesser duration of the treatment (e.g., less than 8 weeks) is also contemplated.

The treatment method according to this aspect of the invention does not include administration of any interferon. The treatment may or may not include administration of ribavirin; preferably the treatment further comprises administering ribavirin to the patient.

The patient being treated according to this aspect of the invention can be a treatment naïve patient, a treatment experienced patient, including, but not limited to, a relapser, an interferon partial responder, an interferon null responder, or a patient unable to take interferon. The patient may be infected with, for example and without limitation. HCV genotype 1, such as HCV genotype 1a or HCV genotype 1b; or HCV genotype 2 or 3. The treatment according to this aspect of the technology may also be effective against other HCV genotypes.

The DAAs used in this aspect of the invention can be administered around the same time or at different times, and can be co-formulated in a single formulation or formulated in different compositions. Other than the HCV protease inhibitor co-administered with ritonavir, the other DAA(s) can be selected, for example and without limitation, from HCV protease inhibitors. HCV polymerase inhibitors, or HCV NS5A inhibitors For instance, the combination of two or more DAAs can be a combination of at least one HCV protease inhibitor and at least one HCV polymerase inhibitor (e.g., a combination of at least one HCV protease inhibitor and at least one non-nucleoside polymerase inhibitor, or a combination of at least one HCV protease inhibitor and at least one nucleoside or nucleotide polymerase inhibitor, or a combination of at least one HCV protease inhibitor, at least one nucleoside or nucleotide polymerase inhibitor and at least one non-nucleoside inhibitor). For another instance, the combination of two or more DAAs can be a combination of at least one HCV protease inhibitor and at least one HCV NS5A. inhibitor. For still another instance, the combination of two or more DAAs can be a. combination of at least one HCV protease inhibitor, at least one HCV polymerase inhibitor, and at least one HCV NS5A inhibitor. For another instance, the combination of two or more DAAs can be to combination of at least two HCV protease inhibitors.

In one example of this aspect of the invention, the combination of two or more DAAs is a combination of Compound 1 (or to salt thereof) and Compound 2 (or a salt thereof). Compound 1 (or a salt thereof) preferably is co-formulated with ritonavir.

In another example, the combination of two or more DAAs is a combination of Compound 1 (or a salt thereof) and Compound 3 (or a salt thereof). Compound 1 (or a salt thereof) preferably is co-formulated with ritonavir.

In still another example, the combination of two or more DAAs is a combination of Compound 1 (or a salt thereof) and Compound 4 (or a salt thereof). Compound 1 (or a salt thereof) preferably is co-formulated with ritonavir.

In a further example, the combination of two or more DAAs is a combination of Compound 1 (or a salt thereof), Compound 2 (or a salt thereof) and Compound 4 (or a salt thereof). Compound 1 (or a salt thereof) preferably is co-formulated with ritonavir. Also preferably, Compound 1 (or a salt thereof), ritonavir and Compound 4 (or a salt thereof) are co-formulated in a single composition. For example, solid dosage formulations of ritonavir with another HCV protease inhibitor and/or anti-HCV agent can be prepared using melt-extrusion or other solid dispersion technologies as described in U.S. Patent Application Publication Nos. 2005/0084529 and 2011/0312973.

In yet another example, the combination of two or more DAAs is a combination of Compound 1 (or a salt thereof), Compound 3 (or a salt thereof) and Compound 4 (or a salt thereof). Compound 1 preferably is co-formulated with ritonavir. Also preferably, Compound 1 (or a salt thereof), ritonavir and Compound 4 (or a salt thereof) are co-formulated in a single composition.

In still another example, the combination of two or more DAAs includes mericitabine and danoprevir. Danoprevir preferably is co-formulated with ritonavir. For example, danoprevir and ritonavir can be co-formulated using melt-extrusion or other solid dispersion technologies as described in U.S. patent application Ser. No. 13/492,211.

In still another example, the method comprises administering 100 or 200 mg Compound 1 together with 100 mg ritonavir once daily, and 25 mg compound 4 once daily.

In yet another example, the method comprises administering 150 mg or 250 mg Compound 1 together with 100 mg ritonavir once daily, and 400 mg Compound 2 twice daily.

In another example, the method comprises administering 150 mg Compound 1 together with 100 mg ritonavir once daily, and 400 mg Compound 3 once daily.

In another example, the method comprises administering 150 mg Compound 1 together with 100 mg ritonavir once daily, and 400 mg Compound 3 twice daily.

In another example, the method comprises administering 100 or 150 mg Compound 1 together with 100 mg ritonavir once daily, 25 mg compound 4 once daily, and 400 mg Compound 2 twice daily.

In another example, the method comprises administering 100 or 150 mg Compound 1 together with 100 mg ritonavir once daily, 25 mg compound 4 once daily, and 400 mg Compound 3 twice daily.

If the treatment comprises administering ribavirin, ribavirin can be administered based on patient weight, and for example, 1000 to 1200 mg divided twice daily.

Other DAA(s) can also be included in a treatment regimen according to this aspect of the invention. Preferred HCV protease inhibitors include, but are not limited to, telaprevir (Vertex), boceprevir (Merck), BI-201335 (Boehringer Ingelheim), GS-9451 (Gilead), and BMS-650032 (BMS). Other suitable protease inhibitors include, but are not limited to, ACH-1095 ACH-1625 (Achillion), ACH-2684 (Achillion), AVL-181 (Avila), AVL-192 (Avila), BMS-650032 (BMS), danoprevir (RG7227/ITMN-191, Roche), GS-9132 (Gilead), GS-9256 (Gilead), IDX-136 (Idenix), IDX-316 (Idenix), IDX-320 (Idenix), MK-5172 (Merck), narlaprevir (Schering-Plough Corp), PHX-1766 (Phenomix), TMC-435 (Tibotec), vaniprevir (MK-7009, Merck), VBY708 (Virobay), VX-500 (Vertex), VX-813 (Vertex), VX-985 (Vertex), or a combination thereof.

Preferred non-nucleoside HCV polymerase inhibitors for use in the present invention include, but are not limited to, GS-9190 (Gilead), BI-207127 (Boehringer Ingelheim), and VX-222 (VCH-222) (Vertex & ViraChem). Preferred nucleotide HCV polymerase inhibitors include, but are not limited to, PSI-7977 (Pharmasset), and PSI-938 (Pharmasset). Other suitable and non-limiting examples of suitable HCV polymerase inhibitors include ANA-598 (Anadys), BI-207127 (Boehringer Ingelheim), BILB-1941 (Boehringer Ingelheim), BMS-791325 (BMS), filibuvir, GL59728 (Glaxo), GL60667 (Glaxo), GS-9669 (Gilead), IDX-375 (Idenix), MK-3281 (Merck), tegobuvir, TMC-647055 (Tibotec), VCH-759 (Vertex & ViraChem), VCH-916 (ViraChem), VX-759 (Vertex), GS-6620 (Gilead), IDX-102 (Idenix), IDX-184 (Idenix), INX-189 (Inhibitex), MK-0608 (Merck), RG7128 (Roche), TMC64912 (Medivir), GSK625433 (GlaxoSmithKline), BCX-4678 (BioCryst), ALS-2200 (Alios BioPharma/Vertex), ALS-2158 (Alios BioPharma/Vertex), or a combination thereof. A polymerase inhibitor may be a nucleoside polymerase inhibitor, such as GS-6620 (Gilead), IDX-102 (Idmix), IDX-184 (Idenix), INX-189 (Inhibitex), MK-0608 (Merck), PSI-7977 (Pharmasset), PSI-938 (Pharmasset), RG7128 (Roche), TMC64912 (Medivir). ALS-2200 (Alias BioPharma/Vertex), ALS-2158 (Alios BioPharma/Vertex), or a combination therefore. A polymerase inhibitor may also be a non-nucleoside polymerase inhibitor, such as PF-00868554 (Pfizer), ANA-598 (Anadys), BI-207127 (Boehringer Ingelheim), BILB-1941 (Boehringer Ingelheim), BMS-791325 (BMS), filibuvir, GL59728 (Glaxo), GL60667 (Glaxo), GS-9669 (Gilead), IDX-375 (Idenix), MK-3281 (Merck), tegobuvir, TMC-647055 (Tibotec), VCH-759 (Vertex & ViraChem), VCH-916 (ViraChem), VX-222 (VCH-222) (Vertex & ViraChem), VX-759 (Vertex), or a combination thereof.

Preferred NS5A inhibitors include, but are not limited to, BMS-790052 (BMS) and GS-5885 (Gilead). Non-limiting examples of suitable NS5A inhibitors include GSK62336805 (GlaxoSmithKline), ACH-2928 AZD2836 (Astra-Zeneca), AZD7295 (Astra-Zeneca), BMS-790052 (BMS), BMS-824393 (BMS), GS-5885 (Gilead), PPI-1301 (Presidio), PPI-461 (Presidio) A-831 (Arrow Therapeutics), A-689 (Arrow Therapeutics) or a combination thereof.

Non-limiting examples of suitable cyclophilin inhibitors include alisporovir (Novartis & Debiopharm), NM-811 (Novartis), SCY-635 (Scynexis), or a combination thereof.

Non-limiting examples of suitable HCV entry inhibitors include ITX-4520 (iTherx), ITX-5061 (iTherx), or a combination thereof.

Specific examples of other DAA agents that are suitable for the present invention include, but are not limited to, AP-H005, A-831 (Arrow Therapeutics) (NS5A inhibitor), A-689 (Arrow Therapeutics) (NS5A inhibitor), INX08189 (Inhibitex) (polymerase inhibitor), ITMN-191 (Intermune/Roche) (NS3/4A Protease inhibitor). VBY-376 (Protease Inhibitor) (Virobay), ACH-1625 (Achillion, Protease inhibitor), IDX136 (Idenix, Protease Inhibitor), IDX316 (Idenix, Protease inhibitor), VX-813 (Vertex), SCH 900518 (Schering-Plough), TMC-435 (Tibotec), ITMN-191 (Intermune, Roche), MK-7009 (Merck), IDX-PI (Novartis), R7128 (Roche), PF-868554 (Pfizer) (non-nucleoside polymerase inhibitor), PF-4878691 (Pfizer), IDX-184 (Idenix), IDX-375 (Idenix, NS5B polymerase inhibitor), PPI-461 (Presidio), BILB-1941 (Boehringer Ingelheim), GS-9190 (Gilead), BMS-790052 (BMS), CTS-1027 (Conatus), GS-9620 (Gilead), PF-4878691 (Pfizer), RO5303253 (Roche), ALS-2200 (Alias BioPharma/Vertex), ALS-2158 (Alios BioPharma/Vertex), GSK62336805 (GlaxoSmithKline), or any combinations thereof.

The chemical structures of some of these HCV inhibitors are provided below:

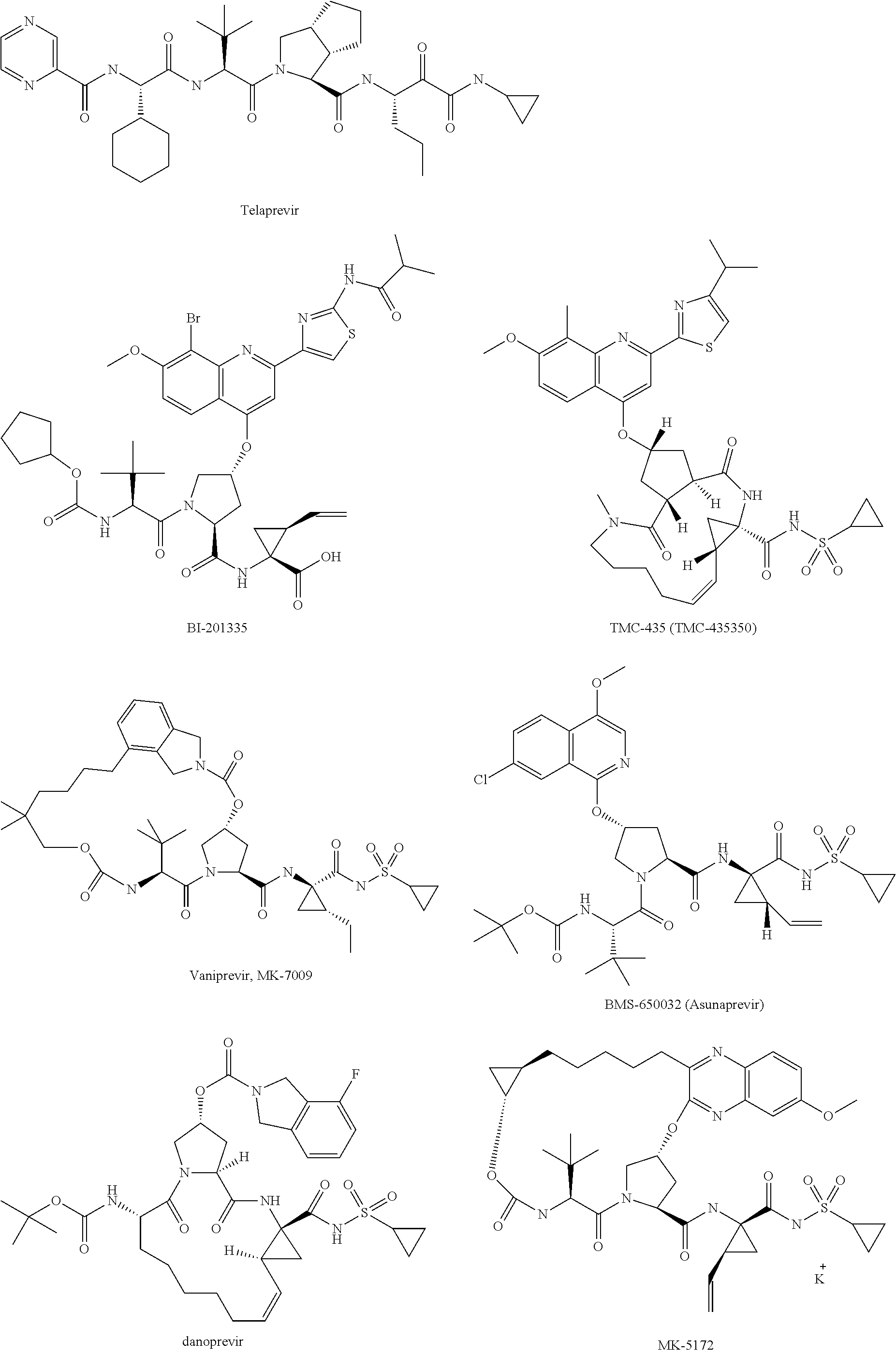

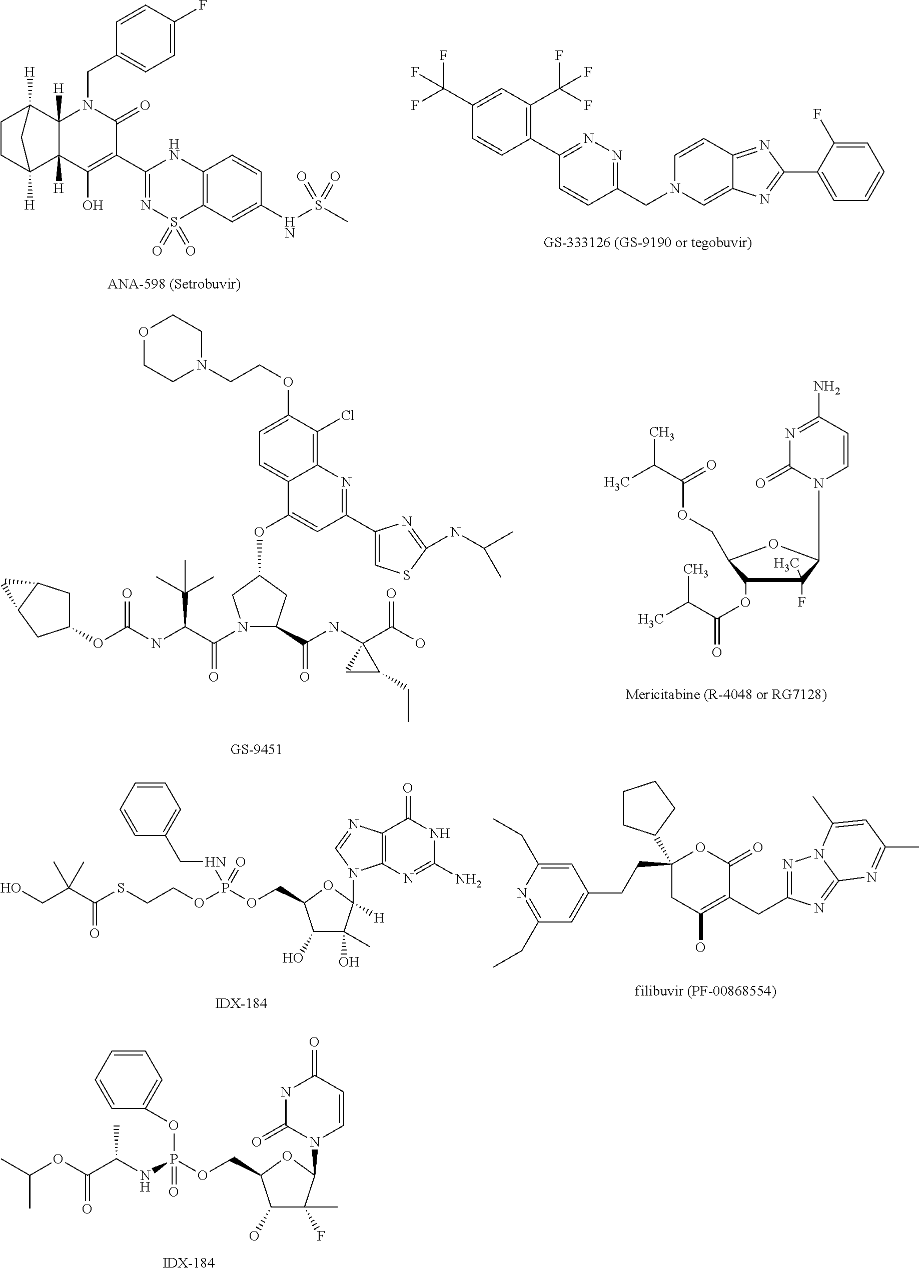

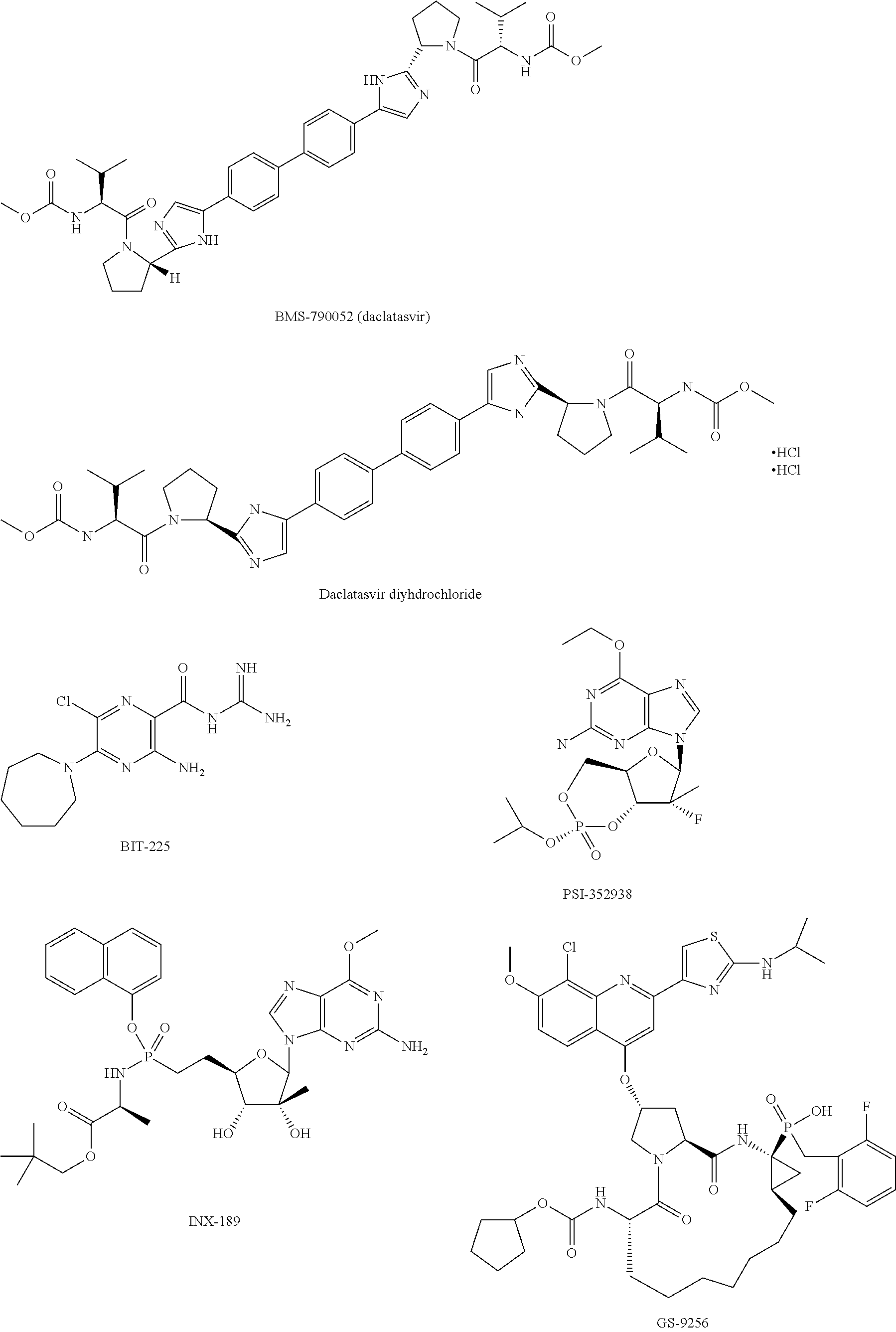

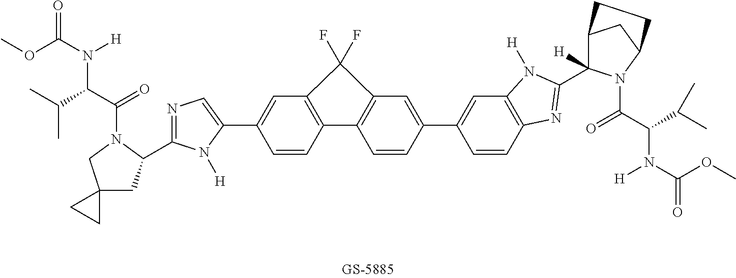

It has also been reported that BMS-791325 has the following structure:

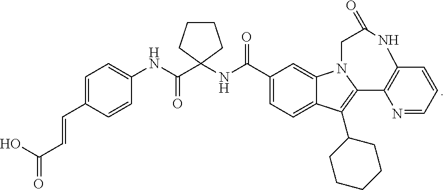 [SMILES: O=C(O)/C=C/c1ccc(NC(=O)C2(NC(=O)c3ccc4c(C5CCCCC5)c5n(c4c3)CC(=O)Nc3cccnc3-5)CCCC2)cc1]

See also publications at http://wwwl.easl.eu/easl2011/program/Posters/Abstract680.htm; and http://clinicaltrials.gov/show/NCT00664625. For GS-5885, see publications at http://www.natap.org/2011/EASL/EASL_{—}68.htm; http://wwwl.easl.eu/easl2011/program/Posters/Abstract1097.htm; and http://clinicaltrials.gov/ct2/show/NCT01353248.

Any HCV inhibitor or DAA described herein encompasses its suitable salt forms when it is used in therapeutic treatments or pharmaceutical formulations.

In any aspect of the invention and each embodiment and example thereof, ritonavir can be readily replaced with cobicistat.

In any aspect of the invention and each embodiment and examples thereof, the testing for the total cholesterol and triglyceride levels can be absent during the treatment, instead of prior to and after the treatment.

It should be understood that the above-described embodiments and the following examples are given by way of illustration, not limitation. Various changes and modifications within the scope of the present invention will become apparent to those skilled in the art from the present description.

### EXAMPLE

### Ritonavir-Containing HCV Treatment Regimens Are Not Associated with Changes in Total Cholesterol and Triglycerides

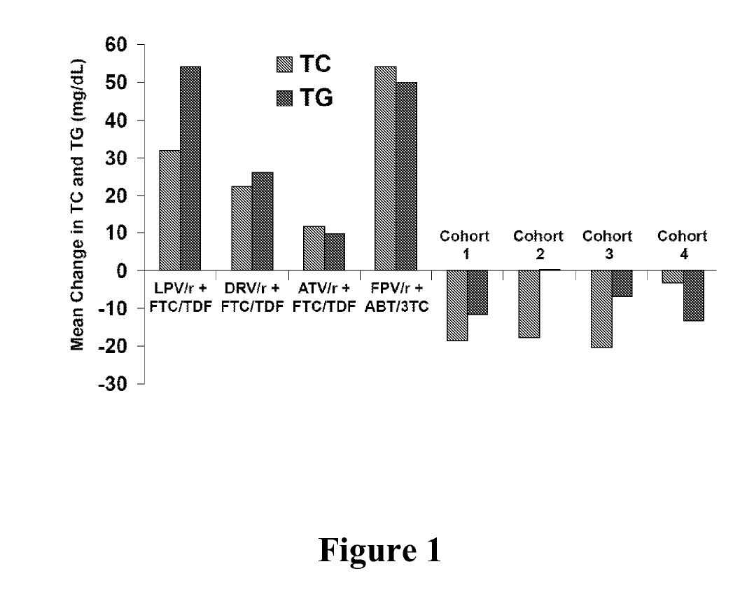

Ritonavir-boosted HIV protease inhibitors are associated with increases in serum lipids. See FIG. 1, where LPV refers to lopinavir, “/r” refers to co-administration with ritonavir (e.g., LPV/r refers to lopinavir co-administered with ritonavir). DRV refers to darunavir, ATV refers to atazanavir, FPV refers to fosamprenavir, FTC refers to emtricitabine, TDF refers to tenofovir disoproxil fumarate, ABT refers to abacavir, 3TC refers to lamivudine, TC refers to total cholesterol, and TG refers to total triglycerides. These increase in cholesterol and triglycerides may be related to inhibition of the proteasome, which is involved in degradation of proteins related to lipid metabolism. However, the impact of ritonavir-boosted HCV treatments on lipid levels has not been studied. The purpose of this Example was to examine the lipid levels of HCV patients during 12 weeks of treatment with ritonavir-containing, interferon-free regimens.

The study contained four cohorts: Cohort 1 included 11 treatment-naïve patients who were subject to 12-week interferon-free treatment comprising Compound 1 (150 mg QD), ritonavir (100 mg QD), Compound 3 (400 mg QD) and ribavirin (weight-based, 1,000-1,200 mg day); Cohort 2 included 19 treatment-nave patients who were subject to 12-week interferon-free treatment comprising Compound 1 (250 mg QD), ritonavir (100 mg QD), Compound 2 (400 mg BID) and ribavirin (weight-based, 1,000-1,200 mg/day); Cohort 3 included 14 treatment-naive patients who were subject to 12-week interferon-free treatment comprising Compound 1 (150 mg QD), ritonavir (100 mg QD), Compound 2 (400 mg BID) and ribavirin (weight-based, 1,000-1,200 mg/day); and Cohort 4 included 17 prior peginterferon/ribavirin non-responders who were subject to 12-week interferon-free treatment comprising Compound 1 (150 mg QD), ritonavir (100 mg QD), Compound 2 (400 mg BID) and ribavirin (weight-based, 1,000-1,200 mg/day). All subjects were followed for 48 weeks after the end of treatment. The enrollment was limited to HCV genotype 1 infection, IL28B SNP rs12979860 CC genotype, absence of HIV or hepatitis B co-infection, and any liver biopsy within the past 3 years being consistent with chronic HCV and no evidence of extensive bridging fibrosis or cirrhosis.

Fasting total cholesterol (TC) and triglycerides (TG) were measured at each study visit. The number of subjects with baseline low or normal TC or TG who shifted to high TC or TG were calculated using NCEP cut-offs. Lipid fractions were not measured.

During the study, there were no Grade 3 or 4 TC or TG elevations; there were no reports of adverse events of elevated TC or TG, and no subject was initiated lipid-lowering agents. As demonstrated in FIG. 1, all 12-week HCV treatments containing a ritonavir-boosted HCV protease inhibitor had no clinically significant impact on total cholesterol or triglycerides in the HCV-infected subjects studied, regardless of whether the subjects are treatment-naïve or non-responders.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A method for treatment of HCV, comprising administering to an HCV patient an effective amount of an HCV protease inhibitor and ritonavir, wherein total cholesterol and triglyceride levels in said patient are not tested prior to and after said treatment.

2. The method of claim 1, wherein said HCV protease inhibitor is Compound 1.

3. The method of claim 1, wherein said HCV protease inhibitor is danoprevir.

4. The method of claim 1, wherein said method further comprises administering to said patient another anti-HCV agent, wherein said another HCV agent is selected from an HCV NS5A inhibitor. an HCV polymerase inhibitor, an HCV entry inhibitor, a cyclophilin inhibitor, a CD81 inhibitor, or an internal ribosome entry site inhibitor.

5. The method of claim 2, wherein said method further comprises administering to said patient another anti-HCV agent, wherein said another HCV agent is selected from an HCV NS5A inhibitor or an HCV polymerase inhibitor.

6. The method of claim 5, wherein said method comprises administering said HCV protease inhibitor and ritonavir to said patient at least once a day for no more than 24 weeks, and wherein said treatment does not include administering interferon to said patient.

7. The method of claim 5, wherein said method comprises administering said HCV protease inhibitor and ritonavir to said patient at least once a day for no more than 12 weeks, and wherein said treatment does not include administering interferon to said patient.

8. The method of claim 5, said method comprises administering said HCV protease inhibitor and ritonavir to said patient at least once a day for 12 weeks, and wherein said treatment does not include administering interferon to said patient.

9. The method according to one of claims 6-8, wherein said another anti-HCV agent is Compound 2.

10. The method according to one of claims 6-8, wherein said another anti-HCV agent is Compound 3.

11. The method according to one of claims 6-8, wherein said another anti-HCV agent is Compound 4.

12. The method according to one of claims 6-11, wherein aid method further comprising administering ribavirin to said patient.

13. The method of claim 3, wherein said method further comprises administering to said patient another anti-HCV agent, wherein said another HCV agent is selected from an HCV NS5A inhibitor or an HCV polymerase inhibitor.

14. The method of claim 13, wherein said method comprises administering said HCV protease inhibitor and ritonavir to said patient at least once a day for no more than 24 weeks, and wherein said treatment does not include administering interferon to said patient.

15. The method of claim 13, wherein said method comprises administering said HCV protease inhibitor and ritonavir to said patient at least once a day for no more than 12 weeks, and wherein said treatment does not include administering interferon to said patient.

16. The method of claim 13, said method comprises administering said HCV protease inhibitor and ritonavir to said patient at least once a day for 12 weeks, and wherein said treatment does not include administering interferon to said patient.

17. The method according to one of claims 13-16, wherein said another anti-HCV agent is mericitabine.

18. The method according to one of claims 13-17, wherein aid method further comprising administering ribavirin to said patient.

19. A method for treatment of HCV, comprising administering to an HCV patient an effective amount of an HCV protease inhibitor and cobicistat, wherein total cholesterol and triglyceride levels in said patient are not tested prior to and after said treatment.

20. The method of claim 19, wherein said treatment does not include administering interferon to said patient.
